(19) United States
(12) Patent Application Publication
(10) Pub. No.: US 2025/0232859 A1
(43) Pub. Date: Jul. 17, 2025

(54) METHODS, SYSTEMS, AND COMPUTER PROGRAM PRODUCTS FOR MANAGING MULTIPLE DRUG PRODUCT PACKAGING SYSTEMS USING A COMMON INTERFACE

(71) Applicant: PARATA SYSTEMS, LLC, Durham, NC (US)

(72) Inventors: Benjamin Heber Clayton, Fuquay-Varina, NC (US); Brian Thomas Bishop, Wonder Lake, IL (US); Richard Eckert, Lindenhurst, IL (US); Joseph Dean Hans, Helenville, WI (US)

(21) Appl. No.: 19/021,525

(22) Filed: Jan. 15, 2025

Related U.S. Application Data
(60) Provisional application No. 63/621,185, filed on Jan. 16, 2024.

(51) Int. Cl. G16H 20/10; G06F 9/54

(52) U.S. Cl. CPC G16H 20/10; G06F 9/541

(57) ABSTRACT

A method includes performing, by a processor: generating a packaging order for a drug product at a host; communicating the packaging order to a translation driver; translating the packaging order into a format specific to a drug product packaging system; and communicating the translated packaging order to a hardware controller associated with the drug product packaging system.

### CROSS-REFERENCE TO RELATED APPLICATION

This application claims priority to U.S. Provisional Patent Application No. 63/621,185, filed on Jan. 16, 2024, the disclosure of which is incorporated herein by reference in its entirety.

### BACKGROUND

The present disclosure relates generally to the dispensing of drug products, and, in particular, to methods, systems, and computer program products for managing multiple drug product packaging systems.

Drug product packaging systems may be used in facilities, such as pharmacies, hospitals, long term care facilities, and the like to dispense medications to fill prescriptions. These drug product packaging systems may include systems designed to package medications in various container types including vials, blister cards, and strip packaging. Strip packaging is a type of packaging wherein medications are packaged in individual pouches for administration on a specific date and, in some cases, at a specific time. Typically, these pouches are removably joined together and often provided in rolls. The pouches can be separated from the roll when needed. Blister cards may be similarly organized according to the times of administration of the medication contained therein. A facility may have multiple drug product packaging systems in operation. The host control application that manages the fulfillment of prescriptions by way of the various drug product packaging systems, however, are typically highly customized to each unique drug product packaging system hardware manufacturer or model. As a result, a host control application may need to be modified and re-released to support new drug product packaging system models and/or to address firmware changes to existing drug product packaging models.

### SUMMARY

In some embodiments of the inventive concept, a method comprises, performing by a processor: generating a packaging order for a drug product at a host; communicating the packaging order to a translation driver; translating the packaging order into a format specific to a drug product packaging system; and communicating the translated packaging order to a hardware controller associated with the drug product packaging system.

In other embodiments, the format is a Transmission Control Protocol/Internet Protocol (TCP/IP) format or a serial format.

In still other embodiments, the format supports an event driven driver architecture between the translation driver and the drug product packaging system.

In still other embodiments, the format supports a polling driven driver architecture between the translation driver and the drug product packaging system.

In still other embodiments, the packaging order identifies label information for a drug product package produced by the drug product packaging system according to the packaging order.

In still other embodiments, the packaging order identifies the drug product for packaging using the drug product packaging system.

In still other embodiments, the packaging order further identifies a source location of the drug product in the drug product packaging system.

In still other embodiments, the packaging order is a first packaging order, the drug product is a first drug product, the drug product packaging system is a first drug product packaging system, the translation driver is a first translation driver, the format is a first format, and the hardware controller is a first hardware controller; and translating the first packaging order comprises translating the first packaging order using the first translation driver, the method further comprising: generating a second packaging order for a second drug product at the host; communicating the second packaging order to a second translation driver; translating the second packaging order using the second translation driver into a second format specific to a second drug product packaging system; and communicating the translated second packaging order to a second hardware controller associated with the second drug product packaging system.

In still other embodiments, the first format and the second format are different.

In still other embodiments, a first driver architecture between the host and the first drug product packaging system is different than a second driver architecture between the host and a second drug product packaging system.

In some embodiments of the inventive concept, a system comprises a processor and a memory coupled to the processor and comprising computer readable program code embodied in the memory that is executable by the processor to perform operations comprising: generating a packaging order for a drug product at a host; communicating the packaging order to a translation driver; translating the packaging order into a format specific to a drug product packaging system; and communicating the translated packaging order to a hardware controller associated with the drug product packaging system.

In further embodiments, the format is a Transmission Control Protocol/Internet Protocol (TCP/IP) format or a serial format.

In still further embodiments, the format supports an event driven driver architecture between the translation driver and the drug product packaging system.

In still further embodiments, the format supports a polling driven driver architecture between the translation driver and the drug product packaging system.

In still further embodiments, the packaging order identifies label information for a drug product package produced by the drug product packaging system according to the packaging order.

In still further embodiments, the packaging order identifies the drug product for packaging using the drug product packaging system.

In still further embodiments, the packaging order further identifies a source location of the drug product in the drug product packaging system.

In still further embodiments, the packaging order is a first packaging order, the drug product is a first drug product, the drug product packaging system is a first drug product packaging system, the translation driver is a first translation driver, the format is a first format, and the hardware controller is a first hardware controller; and translating the first packaging order comprises translating the first packaging order using the first translation driver, the operations further comprising: generating a second packaging order for a second drug product at the host; communicating the second packaging order to a second translation driver; translating the second packaging order using the second translation driver into a second format specific to a second drug product packaging system; communicating the translated second packaging order to a second hardware controller associated with the second drug product packaging system.

In still further embodiments, the first format and the second format are different.

In still further embodiments, a first driver architecture between the host and the first drug product packaging system is different than a second driver architecture between the host and a second drug product packaging system.

In some embodiments of the inventive concept, a computer program product comprises: a non-transitory computer readable storage medium comprising computer readable program code embodied in the medium that is executable by a processor to perform operations comprising: generating a packaging order for a drug product at a host; communicating the packaging order to a translation driver; translating the packaging order into a format specific to a drug product packaging system; and communicating the translated packaging order to a hardware controller associated with the drug product packaging system.

In other embodiments, the format is a Transmission Control Protocol/Internet Protocol (TCP/IP) format or a serial format.

In still other embodiments, the format supports an event driven driver architecture between the translation driver and the drug product packaging system.

In still other embodiments, the format supports a polling driven driver architecture between the translation driver and the drug product packaging system.

In still other embodiments, the packaging order identifies label information for a drug product package produced by the drug product packaging system according to the packaging order.

In still other embodiments, the packaging order identifies the drug product for packaging using the drug product packaging system.

In still other embodiments, the packaging order further identifies a source location in the drug product packaging system of the drug product for packaging using the drug product packaging system.

In still other embodiments, the packaging order is a first packaging order, the drug product is a first drug product, the drug product packaging system is a first drug product packaging system, the translation driver is a first translation driver, the format is a first format, and the hardware controller is a first hardware controller; and translating the first packaging order comprises translating the first packaging order using the first translation driver, the operations further comprising: generating a second packaging order for a second drug product at the host; communicating the second packaging order to a second translation driver; translating the second packaging order using the second translation driver into a second format specific to a second drug product packaging system; and communicating the translated second packaging order to a second hardware controller associated with the second drug product packaging system.

In still other embodiments, the first format and the second format are different.

In still other embodiments, a first driver architecture between the host and the first drug product packaging system is different than a second driver architecture between the host and a second drug product packaging system.

It is noted that aspects described with respect to one embodiment may be incorporated in different embodiments although not specifically described relative thereto. That is, all embodiments and/or features of any embodiments can be combined in any way and/or combination. Moreover, other methods, systems, articles of manufacture, and/or computer program products according to embodiments of the inventive subject matter will be or become apparent to one with skill in the art upon review of the following drawings and detailed description. It is intended that all such additional systems, methods, articles of manufacture, and/or computer program products be included within this description, be within the scope of the present inventive subject matter, and be protected by the accompanying claims. It is further intended that all embodiments disclosed herein can be implemented separately or combined in any way and/or combination.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including a packaging system management server for managing multiple drug product packaging systems using a common interface in accordance with some embodiments of the inventive concept;

FIG. 2 illustrates a data processing system that may be used to implement the packaging system management server of FIG. 1 in accordance with some embodiments of the inventive concept;

FIG. 3 is a block diagram that illustrates a software/hardware architecture for use in a packaging system management server for managing multiple drug product packaging systems using a common interface in accordance with some embodiments of the inventive concept;

FIG. 4 is a block diagram that illustrates control of multiple drug product packaging systems using multiple translation drivers in accordance with some embodiments of the inventive concept; and

FIG. 5 is a flowchart diagram that illustrates operations for managing multiple drug product packaging systems using a common interface in accordance with some embodiments of the inventive concept.

### DETAILED DESCRIPTION

In the following detailed description, numerous specific details are set forth to provide a thorough understanding of embodiments of the present disclosure. However, it will be understood by those skilled in the art that the present invention may be practiced without these specific details. In some instances, well-known methods, procedures, components and circuits have not been described in detail so as not to obscure the present disclosure. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “data processing facility” includes, but it is not limited to, a hardware element, firmware component, and/or software component. A data processing system may be configured with one or more data processing facilities.

The term “drug product packaging system,” as used herein, refers to any type of pharmaceutical dispensing system including, but not limited to, automated systems that fill vials, containers, pouch, or the like with drug product, and semi-automated systems that fill vials, containers, pouches or the like with drug product.

The terms “pharmaceutical” and “medication,” as used herein, are interchangeable and refer to medicaments prescribed to patients.

The term “drug product” refers to any type of medicament that can be packaged within a vial, container, pouch, or the like by automated and semi-automated drug product packaging systems including, but not limited to, pills, capsules, tablets, caplets, gel caps, lozenges, and the like.

Some embodiments of the inventive concept stem from a realization that in a typical facility or network, such as a such as pharmacy, hospital, long term care facility, and the like multiple drug product packaging systems are frequently managed together. The host control applications that manage the various drug product packaging systems, however, are typically highly customized to each unique drug product packaging system hardware manufacturer or model. When a new drug product packaging system is installed and/or when an existing drug product packaging system is modified, such as the addition of new firmware or hardware, the host control application may need to be modified and reinstalled to accommodate the new drug product packaging system or modified existing drug product packaging system. Some embodiments of the inventive concept may provide a modular translation driver that is configurable to translate packaging orders from a host core service application into a format specific to a drug product packaging system. The translated packaging order may then be communicated to a hardware controller associated with the drug product packaging system. Thus, rather than modify and reinstall the host core service application each time a new drug product packaging system is added to the facility or network, or a drug product packaging system is modified with new firmware and/or hardware, a new modular translation driver may be added as an interface between the host core service application and the hardware controller of the new or modified drug product packaging system. In this way, the use of modular translation drivers that are tailored to the specific communication formats of the different drug product packaging systems may allow a host core service application to serve as a hub in a one-to-many relationship between itself and the different drug product packaging systems.

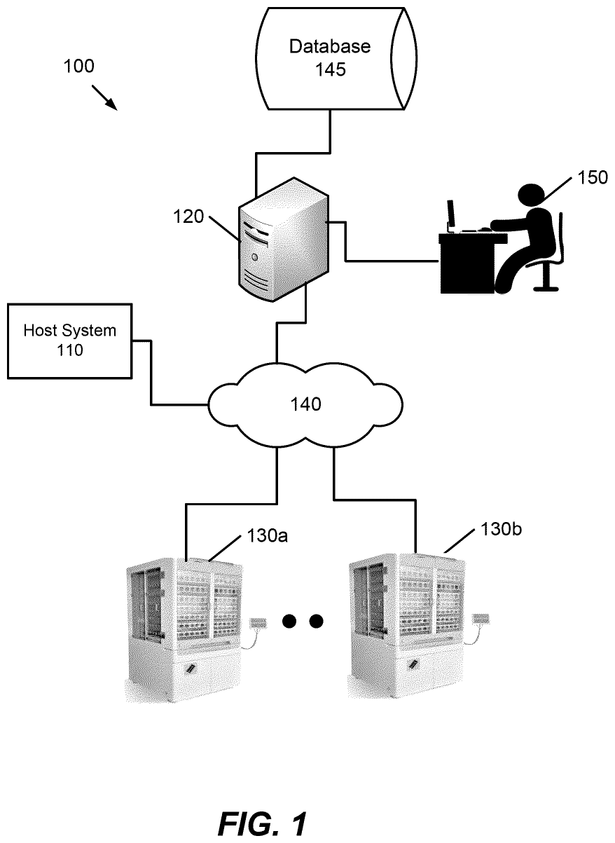

Referring to FIG. 1, a communication network 100, in accordance with some embodiments of the present inventive concept, comprises a host system 110, a packaging system management server 120, and multiple drug product packaging systems 130a and 130b that are coupled via a network 140 as shown. The network 140 may be a global network, such as the Internet or other publicly accessible network. Various elements of the network may be interconnected by a wide area network, a local area network, an Intranet, and/or other private network, which may not be accessible by the general public. Thus, the network 140 may represent a combination of public and private networks or a virtual private network (VPN).

The host system 110 may be configured to manage and fill prescriptions for customers. The host system 110 may be associated with a variety of types of facilities, such as pharmacies, hospitals, long term care facilities, and the like. The packaging system management server 120 may be configured to manage the operation of the drug product packaging systems 130a and 130b. For example, the packaging system management server 120 may be configured to receive packaging orders from the pharmacy host system 110 and to identify which of the drug product packaging systems 130a and 130b should be used to package particular individual orders or batches of orders. U.S. Patent Publication No. 2022/0129819 describes example embodiments for assigning packaging orders to different drug product packaging systems, the disclosure of which is hereby incorporated herein by reference. In addition, the packaging system management server 120 may be configured to manage the operations of the drug product packaging systems 130a and 130b. For example, the packaging system management server 120 may be configured to manage the inventory of drug product available through each of the drug product packaging systems 130a and 130b, to manage the drug product dispensing canisters assigned or registered to one or more of the drug product packaging systems 130a and 130b, and/or to manage reports regarding the status (e.g., assignment, completion, etc.) of packaging orders, drug product inventory, order billing, and the like. A user 150, such as a pharmacist or pharmacy technician, may communicate with the packaging system management server 120 using any suitable computing device via a wired and/or wireless connection. Although the user 150 is shown communicating with the packaging system management server 120 via a direct connection in FIG. 1, it will be understood that the user 150 may communicate with the packaging system management server 120 via one or more network connections. The user 150 may interact with the packaging system management server 120 to approve or override various recommendations made by the packaging system management server 120 in operating the drug product packaging systems 130a and 130b. The user 150 may also initiate the running of various reports as described above for the drug product packaging systems 130a and 130b. Although only two drug product packaging systems 130a and 130b are shown in FIG. 1, it will be understood that more than two drug product packaging systems may be managed using a common database management system in accordance with various embodiments of the inventive concept.

The packaging system management server 120 may use a common database management system to manage a database 145 that contains records corresponding to drug product packaging systems 130a and 130b as described in U.S. Pat. No. 10,492,987, the disclosure of which is hereby incorporated herein by reference. According to some embodiments of the inventive concept, a common database management system and database may be used to store records for all of the drug product packaging systems of the same type or, in other embodiments, a common database management system and database may be used to store records for drug product packaging systems of different types. For example, some drug product packaging systems may be configured to package one or more drug products in pouches formed in a container strip. Other drug product packaging systems may be configured to package one or more drug products in a vial. Thus, a common database management system and database may be used for all of the drug product packaging systems that package one or more drug products in a vial and a common database management system and database may be used for all of the drug product packaging systems that package one or more drug products in pouches formed in a container strip. In accordance with other embodiments, a common database management system may be configured to store records for both a vial type packaging system and a strip type packaging system in the same database. In some embodiments, the database 145 may be a relational database, such as a Structured Query Language (SQL) or DB2 database. It will be understood, however, that embodiments of the inventive concept are not limited in their application to a relational database model as other database models, such as, but not limited to, a flat database model, a hierarchical database model, a network database model, an object-relational database model, and a star schema database model may also be used. It will be appreciated that in accordance with various embodiments of the inventive concept, the packaging system management server 120 may be implemented as a single server, separate servers, or a network of servers either co-located in a server farm, for example, or located in different geographic regions.

In other embodiments, the packaging system management server 120 may use a separate database management system to manage records for one or more of the drug product packaging systems 130a and 130b.

Although FIG. 1 illustrates an example communication network for managing multiple drug product packaging systems using a common interface, it will be understood that embodiments of the inventive subject matter are not limited to such configurations, but are intended to encompass any configuration capable of carrying out the operations described herein.

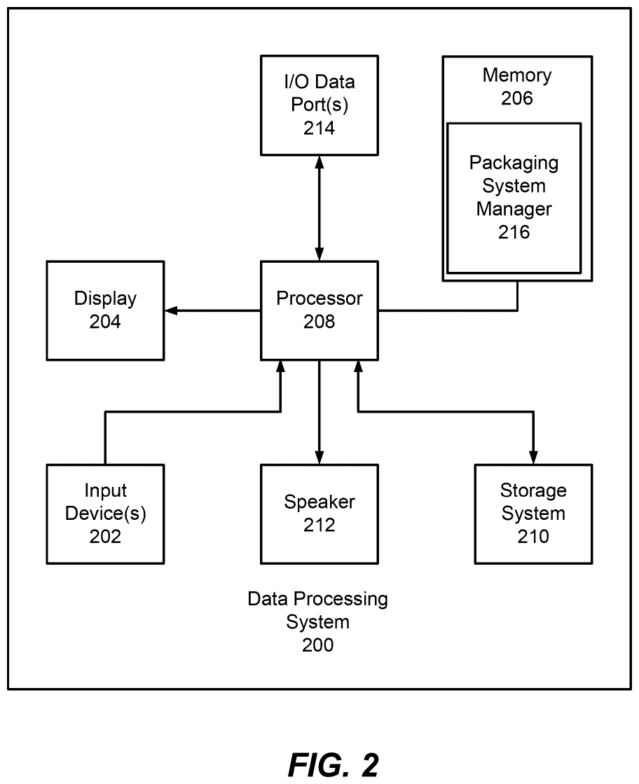

Referring now to FIG. 2, a data processing system 200 that may be used to implement the packaging system management server 120 of FIG. 1, in accordance with some embodiments of the inventive concept, comprises input device(s) 202, such as a keyboard, keypad, or scanner (e.g., barcode, RFID, etc.), a display 204, and a memory 206 that communicate with a processor 208. The data processing system 200 may further include a storage system 210, a speaker 212, and an input/output (I/O) data port(s) 214 that also communicate with the processor 208. The storage system 210 may include removable and/or fixed media, such as floppy disks, ZIP drives, hard disks, or the like, as well as virtual storage, such as a RAMDISK. The I/O data port(s) 214 may be used to transfer information between the data processing system 200 and another computer system or a network (e.g., the Internet). The memory 206 may be configured with a packaging system manager module 216 that may provide functionality that may include, but is not limited to, managing multiple drug product packaging systems using a common interface in accordance with some embodiments of the inventive concept.

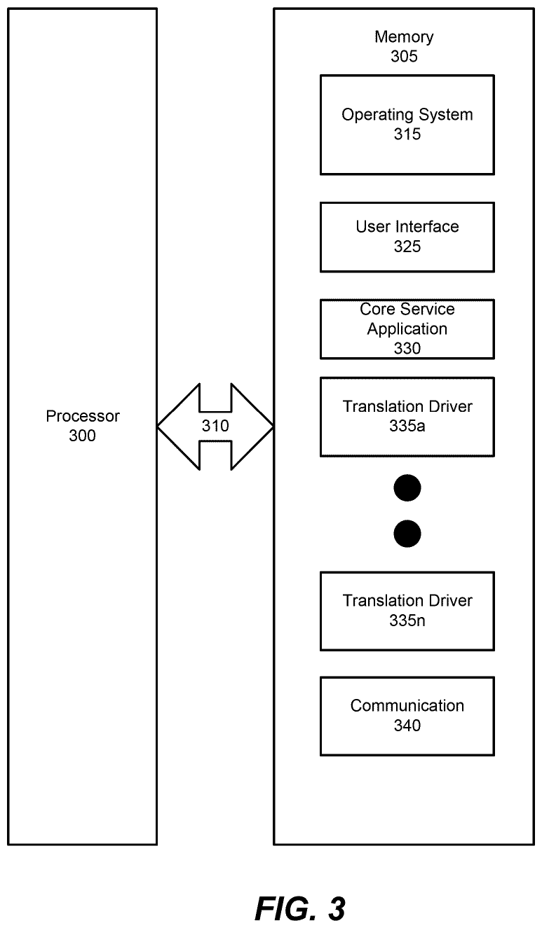

FIG. 3 illustrates a processor 300 and memory 305 that may be used in embodiments of data processing systems, such as the packaging system management server 120 of FIG. 1 and the data processing system 200 of FIG. 2, respectively, for managing multiple drug product packaging systems using a common interface in accordance with some embodiments of the inventive concept. The processor 300 communicates with the memory 305 via an address/data bus 310. The processor 300 may be, for example, a commercially available or custom microprocessor. The memory 305 is representative of the one or more memory devices containing the software and data used for managing multiple drug product packaging systems using a common interface in accordance with some embodiments of the inventive concept. The memory 305 may include, but is not limited to, the following types of devices: cache, ROM, PROM, EPROM, EEPROM, flash, SRAM, and DRAM.

As shown in FIG. 3, the memory 305 may contain three or more categories of software and/or data: an operating system 315, a user interface 325, a core service application interface 330, one or more translation drivers 335a, . . . , 335n, and a communication module 340. In particular, the operating system 315 may manage the data processing system's software and/or hardware resources and may coordinate execution of programs by the processor 300. The user interface 325 may be used to receive from the packaging system management server 120, for example, canister management and/or tray management information for the various drug product packaging servers 130a, 130b as well as configuration settings for one or more translation drivers 335a, . . . , 335n, which may include one or more hardware and/or software parameters. These configuration settings may be pushed out to the translation drivers 335a, . . . , 335n that are installed on the host system server 110 and/or communicate with the host system server 110. The core service application 330 represents the host control application that manages the fulfillment of prescriptions by way of the various drug product packaging systems 335a, . . . , 335n. Each of the translation drivers 335a, . . . , 335n may be configurable to translate packaging orders from the core service application 330 into a format specific to a drug product packaging system 130a, 130b. The translated packaging order may then be communicated to a hardware controller associated with the drug product packaging system 130a, 130b using the communication module 340, which may support a variety of communication formats and protocols, including, but not limited to, the Transmission Control Protocol/Internet Protocol (TCP/IP) format and the serial format, which may include, but is not limited to, EIA, RS-232, and RS-485.

Although FIG. 3 illustrates hardware/software architectures that may be used in data processing systems, such as the packaging system management server 120 of FIG. 1 and the data processing system 200 of FIG. 2, respectively, for managing multiple drug product packaging systems using a common interface in accordance with some embodiments of the inventive concept, it will be understood that the present invention is not limited to such a configuration but is intended to encompass any configuration capable of carrying out operations described herein.

Computer program code for carrying out operations of data processing systems discussed above with respect to FIGS. 1-3 may be written in a high-level programming language, such as C and/or C++, for development convenience. In addition, computer program code for carrying out operations of the present invention may also be written in other programming languages, such as, but not limited to, interpreted languages, such as Python or Java. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the host system server 110 of FIG. 1, the data processing system 200 of FIG. 2, and the hardware/software architecture of FIG. 3 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.”

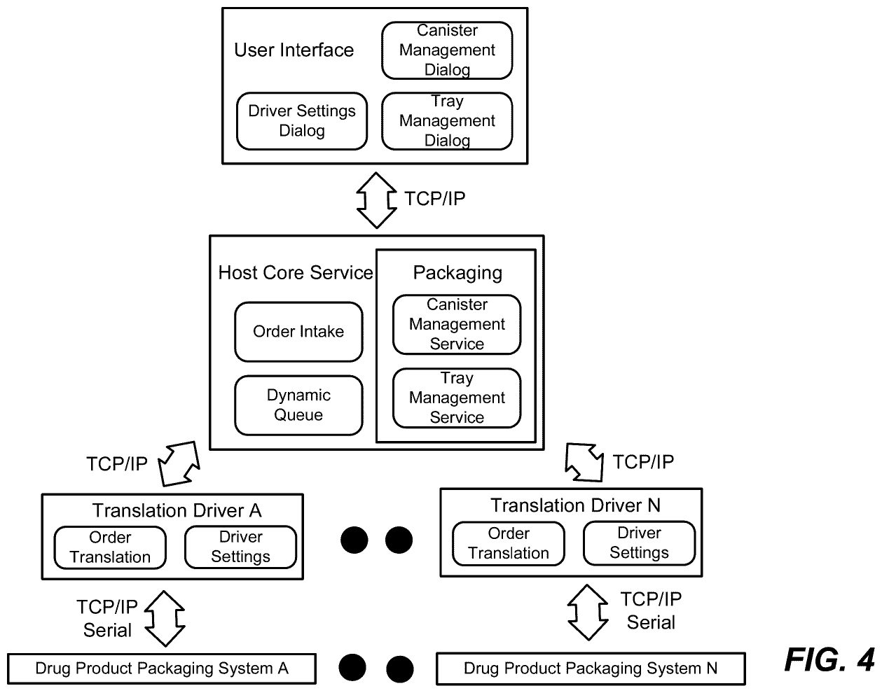

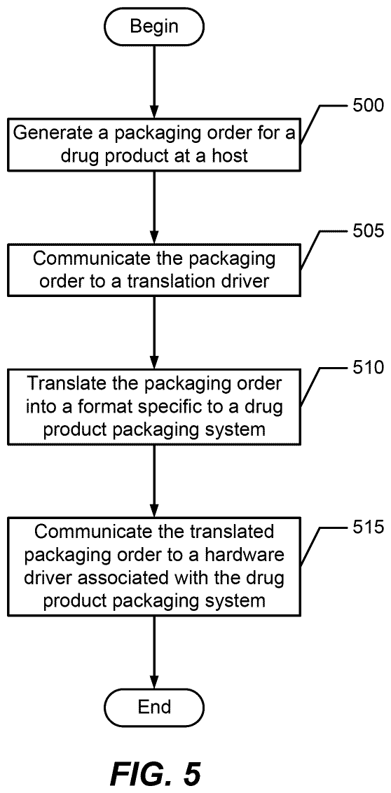

The data processing apparatus of FIGS. 1-3 may be used to facilitate the management of multiple drug product packaging systems using a common interface according to various embodiments described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 206 coupled to the processor 208 and the memory 305 coupled to the processor 300 include computer readable program code that, when executed by the respective processors, causes the respective processors to perform operations including one or more of the operations described herein with respect to FIGS. 4 and 5.

FIG. 4 is a block diagram that illustrates control of multiple drug product packaging systems using multiple translation drivers in accordance with some embodiments of the inventive concept. As shown in FIG. 4, the host core service application may receive canister and/or tray management information by way of the user interface. The canister and/or tray information may be used to identify the source location of a drug product in a drug product packaging system and may, therefore, be included in a packaging order destined for the drug product packaging system. The translation driver settings may be pushed out to the translation drivers that are installed on the packaging management system server 120 and/or communicate with the packaging management system server 120. The host core service application may accept drug product packaging orders for fulfillment by way of an order intake portal and these orders may be added to a dynamic queue for assignment to one of the drug product packaging systems. Example techniques for assigning the orders to the different drug product packaging systems are described, for example, in U.S. Patent Publication No. 2022/0129819. The packaging order may include canister and/or tray information for the destination drug product packaging system to identify a source location of the drug product in the drug product packaging system. The packaging order may further include label information for the drug product package along with an identity of the drug product being packaged using the destination drug product packaging system. As shown in FIG. 4, multiple translation drivers A through N are used to support A through N different drug product packaging systems. Each of the translation drivers is configured to translate the packaging order into a format specific to its corresponding drug product packaging system and then communicate the translated packaging order to a hardware controller associated with the corresponding drug product packaging system. In accordance with different embodiments of the inventive concept, the formats may include the TCP/IP format and the serial format. Moreover, the formats may support different types of driver architectures between the packaging management system server 120 and the drug product packaging systems. For example, a translation driver may use a format that supports various types of driver architectures, such as an event driven driver architecture between the translation driver and the drug product packaging system(s) or a polling driven driver architecture between the translation driver and the drug product packaging system(s). A different translation driver will be created for each unique set of driver requirements associated with the individual drug product packaging systems. Thus, each of the translation drivers uses a unique format specific to the drug product packaging system associated therewith. It will be understood, however, that the host core service application by way of the packaging management system server 120 may communicate with multiple drug product packaging systems using the same format via a single translation driver supporting that format.

FIG. 5 is a flowchart diagram that illustrates operations for managing multiple drug product packaging systems using a common interface in accordance with some embodiments of the inventive concept. Operations begin at block 500 where the core service application 330 generates a packaging order for a drug product at the host system 110. The packaging order is communicated to a translation driver at block 505 and the translation driver 335a, . . . , 335n translates the packaging order into a format specific to the drug product packaging system to which the order is destined. To facilitate the translation operation, communication between the core service application 330 and the translation driver 335a, . . . , 335n may use common or a defined vocabulary of terms that is drug product packaging system independent for describing the packaging order. The translation driver 335a, . . . , 335n may then translate this common order into the specific command and using the specific driver format associated with the destination drug product packaging system. The communication module 340 is used to communicate the translated packaging order to a hardware controller associated with the destination drug product packaging system at block 515.

As described above, some embodiments of the inventive concept may provide a modular translation driver that can be installed independently of the core service application used to fulfill drug product orders using a drug product packaging system. As a result, rather than having to update and reinstall the core service application every time a new drug product packaging system is installed that has a different driver interface than other drug product packaging systems in the network or facility, which can be costly in terms of coding and maintenance effort, a modular translation driver can be installed for each unique drug product packaging system without the need for modifications to the core service application running on the host system.

### Further Definitions and Embodiments

In the above-description of various embodiments of the present disclosure, aspects of the present disclosure may be illustrated and described herein in any of a number of patentable classes or contexts including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present disclosure may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods, and computer program products according to various aspects of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “include,” “including,” “includes,” “have,” “has,” “having,” or variants thereof when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

It will also be understood that, although the terms first, second, etc. may be used herein to describe various elements, these elements should not be limited by these terms. These terms are only used to distinguish one element from another.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The aspects of the disclosure herein were chosen and described to best explain the principles of the disclosure and the practical application, and to enable others of ordinary skill in the art to understand the disclosure with various modifications as are suited to the particular use contemplated.

1. A method comprising:
performing by a processor:
generating a packaging order for a drug product at a host;
communicating the packaging order to a translation driver;
translating the packaging order into a format specific to a drug product packaging system; and
communicating the translated packaging order to a hardware controller associated with the drug product packaging system.

2. The method of claim 1, wherein the format is a Transmission Control Protocol/Internet Protocol (TCP/IP) format or a serial format.

3. The method of claim 1, wherein the format supports an event driven driver architecture between the translation driver and the drug product packaging system.

4. The method of claim 1, wherein the format supports a polling driven driver architecture between the translation driver and the drug product packaging system.

5. The method of claim 1, wherein the packaging order identifies label information for a drug product package produced by the drug product packaging system according to the packaging order.

6. The method of claim 1, wherein the packaging order identifies the drug product for packaging using the drug product packaging system.

7. The method of claim 6, wherein the packaging order further identifies a source location of the drug product in the drug product packaging system.

8. The method of claim 1, wherein the packaging order is a first packaging order, the drug product is a first drug product, the drug product packaging system is a first drug product packaging system, the translation driver is a first translation driver, the format is a first format, and the hardware controller is a first hardware controller; and
wherein translating the first packaging order comprises translating the first packaging order using the first translation driver, the method further comprising:
generating a second packaging order for a second drug product at the host;
communicating the second packaging order to a second translation driver;
translating the second packaging order using the second translation driver into a second format specific to a second drug product packaging system; and
communicating the translated second packaging order to a second hardware controller associated with the second drug product packaging system.

9. The method of claim 8, wherein the first format and the second format are different.

10. The method of claim 8, wherein a first driver architecture between the host and the first drug product packaging system is different than a second driver architecture between the host and a second drug product packaging system.

11. A system, comprising:
a processor; and
a memory coupled to the processor and comprising computer readable program code embodied in the memory that is executable by the processor to perform operations comprising:
generating a packaging order for a drug product at a host;
communicating the packaging order to a translation driver;
translating the packaging order into a format specific to a drug product packaging system; and
communicating the translated packaging order to a hardware controller associated with the drug product packaging system.

12. The system of claim 11, wherein the format is a Transmission Control Protocol/Internet Protocol (TCP/IP) format or a serial format.

13. The system of claim 11, wherein the format supports an event driven driver architecture between the translation driver and the drug product packaging system.

14. The system of claim 11, wherein the format supports a polling driven driver architecture between the translation driver and the drug product packaging system.

15. The system of claim 11, wherein the packaging order identifies label information for a drug product package produced by the drug product packaging system according to the packaging order.

16. The system of claim 11, wherein the packaging order identifies the drug product for packaging using the drug product packaging system.

17. The system of claim 16, wherein the packaging order further identifies a source location of the drug product in the drug product packaging system.

18. The system of claim 11, wherein the packaging order is a first packaging order, the drug product is a first drug product, the drug product packaging system is a first drug product packaging system, the translation driver is a first translation driver, the format is a first format, and the hardware controller is a first hardware controller; and
wherein translating the first packaging order comprises translating the first packaging order using the first translation driver, the operations further comprising:
generating a second packaging order for a second drug product at the host;
communicating the second packaging order to a second translation driver;
translating the second packaging order using the second translation driver into a second format specific to a second drug product packaging system; and
communicating the translated second packaging order to a second hardware controller associated with the second drug product packaging system.

19. The system of claim 18, wherein the first format and the second format are different.

20. The system of claim 18, wherein a first driver architecture between the host and the first drug product packaging system is different than a second driver architecture between the host and a second drug product packaging system.

21-30. (canceled)
